(19) United States
(12) Patent Application Publication
(10) Pub. No.: US 2024/0016445 A1
(43) Pub. Date: Jan. 18, 2024

(54) NON-INVASIVE METHOD FOR MEASURING A PHYSICAL QUANTITY REPRESENTATIVE OF THE ELASTICITY OF A MATERIAL

(71) Applicants: CENTRE NATIONAL DE LA RECHERCHE SCIENTIFIQUE, PARIS (FR); ECOLE CENTRALE DE LYON, ECULLY (FR); ECOLE NATIONALE DES TRAVAUX PUBLICS DE L'ETAT, VAULX-EN-VELIN (FR)

(72) Inventors: Roberto VARGIOLU, CHARLY (FR); Hassan ZAHOUANI, ECULLY CEDEX (FR); Alexandre BERGHEAU, LYON (FR)

(73) Assignees: CENTRE NATIONAL DE LA RECHERCHE SCIENTIFIQUE, PARIS (FR); ECOLE CENTRALE DE LYON, ECULLY (FR); ECOLE NATIONALE DES TRAVAUX PUBLICS DE L'ETAT, VAULX-EN-VELIN (FR)

(21) Appl. No.: 18/251,927

(22) PCT Filed: Nov. 5, 2021
(86) PCT No.: PCT/EP2021/080851

§ 371 Date: May 5, 2023

(30) Foreign Application Priority Data
Nov. 6, 2020 (FR) 2011409

(51) Int. Cl. A61B 5/00

(52) U.S. Cl. CPC A61B 5/442; A61B 5/0059; A61B 5/0053; A61B 5/7235

(57) ABSTRACT

A non-invasive method for measuring a physical quantity representative of the elasticity of a material, including: determining the phase velocities of the fundamental mode of the various components of a shear wave generated at the surface of the material, along a measurement axis, the set of pairs, each formed by a frequency f, and the phase velocity Vi of the fundamental mode determined for the frequency fi, forming a dispersion curve of the fundamental mode in a measurement direction parallel to the measurement axis, wherein the index i is an order number of the frequency fi and of the phase velocity Vi, and transforming the dispersion curve into a profile of phase velocities as a function of depth, the phase velocity at a given depth being a physical quantity representative of the elasticity of a material at the depth.

### FIELD

The invention relates to a non-invasive method and apparatus for measuring a physical quantity representative of the elasticity of a material.

The invention especially relates to a non-invasive method and apparatus for measuring a physical quantity representative of the elasticity of a viscoelastic and/or deformable material or substrate.

### BACKGROUND

By way of example, the measurement of mechanical properties of the skin such as elasticity is useful, for example, to assist in the diagnosis of some skin diseases, or to measure the effectiveness and consequences of some cosmetic treatments on the skin, or to measure the elasticity of tissue-engineered skin in-vitro (bio-printing), or to measure the effect of a mechanical stimulation of tissue-engineered skin in-vitro (bio-printing).

Such a known method is for example described in the following article: M. Ayadh et al: “Methods for characterizing the anisotropic behavior of the human skin's relief and its mechanical properties in vivo linked to age effects”, IOP Publishing, Surf. Topogr.: Metrol. Prop. 8 (2020) 014002, 19 Mar. 2020. Hereinafter, this article is referred to as “Ayadh2020”.

This known method is advantageous in that it is non-invasive. It is therefore simple to implement on the skin of a patient. This known method allows the Rayleigh velocity at the skin surface to be measured in different directions. The Rayleigh velocity is proportional to a phase velocity representative of the elasticity of the skin.

The skin as well as other viscoelastic or deformable materials may resemble a multilayer substrate in some aspects. The method described in Ayadh2020 only provides information on mechanical properties of the surface layer of the skin, especially the epidermis. On the other hand, no information on the mechanical properties of the layers of the skin located under this surface layer can be obtained using this known method. In particular, this known method does not allow mechanical properties of sub-layers such as the dermis and the hypodermis to be measured.

### SUMMARY

The aim of the invention is therefore to provide a method for measuring a quantity representative of the elasticity of a material which additionally allows a physical quantity representative of the elasticity of the sublayers of the material to be measured.

One object of the invention is therefore such a non-invasive method for measuring a physical quantity representative of the elasticity of a material, this method comprising the following steps of:
a) deforming the material at an impact point using a stimulator to generate a shear wave comprising components at different frequencies, these various components propagating at the surface of the material and causing a displacement of the surface of the material,
b) measuring, using a measurement device, the displacement of the surface of the material over time at at least three measurement points aligned one behind the other along a measurement axis,
   wherein the method also comprises the following steps of:
c) determining the phase velocities of the fundamental mode of the individual components of the shear wave generated, along the measurement axis, from the measurements of the measurement device, the set of pairs, each formed by a frequency fᵢ and the phase velocity Vᵢ of the fundamental mode determined for said frequency fᵢ, forming a dispersion curve of the fundamental mode in a measurement direction parallel to the measurement axis, wherein the index “I” is an order number of the frequency fᵢ and the phase velocity Vᵢ, and
d) transforming said dispersion curve Into a profile of phase velocities as a function of depth, the phase velocity at a given depth being a physical quantity representative of the elasticity of the material at said depth.

Measured materials or substrates include viscoelastic and/or deformable materials and may comprise, for example, human skin, artificial skin, animal skin, including marine animal skin such as fish skin, vegetable or fruit skin. These materials may also comprise synthetic or vegetable leather, as well as some polymers such as those used for organ phantoms for example. The method can also be implemented with textiles or even coatings, such as road surfaces.

Embodiments of the method may comprise one or more of the following characteristics:

1) transforming the dispersion curve into a profile of phase velocities in the measurement direction comprises the following operations of:
o1) converting each frequency fᵢ of the dispersion curve, into a corresponding wavelength λᵢ using the following relationship: λᵢ=Vᵢ/fᵢ, and then
o2) calculating the value of a coefficient α for said measurement direction using the following relationship: α=Zₘₐₓ/λₘₐₓ, where:
   Zₘₐₓ is equal to half the distance separating the two furthest measurement points along the measurement axis, and
   λₘₐₓ is the largest of the wavelengths λᵢ obtained after executing the operation o1) for said measurement direction,
o3) converting each wavelength λᵢ obtained from the operation o1) into a corresponding depth pᵢ using the following relationship: pᵢ=αλᵢ, where α is the coefficient calculated in the operation o2).
2) the method comprises executing steps a), b), c) and d) for at least a first and a second measurement axis angularly offset from each other, said first and second measurement axes passing through the same impact point.
3) step c) of determining the phase velocities of the fundamental mode comprises reiterating the following operations for several different frequencies fᵢ:
   for each measurement point:
      filtering, using a bandpass filter centred on the frequency fᵢ and whose −3 dB bandwidth is between the frequencies fᵢ₋₁ and fᵢ₊₁, the signal u(x,t), measured at the measurement point of coordinate x along the measurement axis by the measurement device, in order to obtain a filtered signal uᵢ(x,t),
      identifying a time instant t_{i,m}(x) at which the filtered signal uᵢ(x,t) passes through its absolute minimum, and then
calculating the velocity Vᵢ of propagation of this minimum along the measurement axis from the times t_{i,m}(x) and the positions x at which this minimum occurs, the velocity Vᵢ thus calculated being the phase velocity of the fundamental mode at said frequency fᵢ.
4) step c) includes:
   an operation of automatically identifying, among the set of frequencies fᵢ, a minimum frequency fₘᵢₙ below which the frequencies fᵢ no longer verify the following condition (1):

$${\sum }_{p = 1}^{{P}_{max}} {( {t}_{i , m} ( {x}_{p} ) - {μ}_{i , 1} {x}_{p} - {μ}_{i , 0} )}^{2} \leq {err}_{max} \qquad [ MATH 1 ]$$

where:
   μ_{i,1} and μ_{i,0} are the coefficients of the straight line, determined by the least squares method, best approximating the points of coordinates (x; t_{i,m}(x)),
   xₚ is equal to the position x of the p-th measurement point counted from the first measurement point closest to the impact point,
   Pₘₐₓ is equal to the number of measurement points distributed along the measurement axis,
   errₘₐₓ is a predetermined constant,
and then only the frequencies fᵢ greater than or equal to said frequency fₘᵢₙ are retained to form the dispersion curve.
5) step c) comprises:
   an operation of automatically identifying, from the set of frequencies fᵢ, a maximum frequency fₘₐₓ above which the frequencies fᵢ no longer verify the condition (1), this operation of automatically identifying the frequency fₘₐₓ being carried out by testing this condition (1) for several frequencies fᵢ, and then
   only the frequencies fᵢ less than or equal to said frequency fₘₐₓ are retained to form the dispersion curve.
6) all frequencies fᵢ are between 1 Hz and 3,000 Hz.

Another object of the invention is a non-invasive apparatus for measuring a physical quantity representative of the elasticity of a material, for implementing the above method, this apparatus including:
a stimulator capable of deforming the material at an impact point to generate a shear wave comprising components at different frequencies, these various components propagating at the surface of the material and causing a displacement of the surface of the material,
a measurement device capable of measuring the displacement of the surface of the material over time at at least three measurement points aligned one behind the other along a measurement axis,
   wherein the apparatus comprises a processing unit capable of:
determining the phase velocities of the fundamental mode of the various components of the shear wave generated, along the measurement axis, from the measurements of the measurement device, the set of pairs, each formed by a frequency fᵢ and the phase velocity Vᵢ of the fundamental mode determined for said frequency fᵢ, forming a dispersion curve of the fundamental mode in a measurement direction parallel to the measurement axis, wherein the index “I” is an order number of the frequency fᵢ and the phase velocity Vᵢ, and
transforming said dispersion curve into a profile of phase velocities as a function of depth, the phase velocity at a given depth being a physical quantity representative of the elasticity of the material at said depth.

Embodiments of the apparatus may comprise one or more of the following characteristics:
1)
   the measurement device comprises an array of sensors each capable of measuring the amplitude of the deformation of the surface of the material at a respective measurement point, said array comprising at least three sensors each measuring the displacement of the surface of the material at three respective measurement points aligned one behind the other along a measurement axis, and
   the apparatus comprises a hinged arm to which the sensor array is mounted, said hinged arm being capable of rotating the sensor array through a predetermined angle about an axis of rotation, in order to align the measurement axis of the sensor array with a first measurement axis and, alternately, with a second measurement axis angularly offset from the first measurement axis.
2)
   the sensor array includes:
   a row of optical sensors that sense light reflected from each measurement point, and
   a microprocessor configured to determine the displacement of the surface of the material at each measurement point lit from the reflected light sensed by the row of optical sensors,
   the measurement device comprises an emitter of a light beam which lights each measurement point aligned along the measurement axis.
3) the stimulator is capable of projecting a jet of fluid onto the surface of the material which causes the material to deform at the impact point.

The measurement method and apparatus according to the invention can be implemented in a variety of applications, in various fields such as health, the pharmaceutical industry, cosmetics, quality control, etc.

Measurements can especially be performed on any type of soft tissue, in vivo or collected for example by biopsy. According to examples, various pathologies can be monitored and analysed. Skin tumour analyses can be performed in vivo or after tissue collection. Collagen pathologies, such as scleroderma or osteogenesis imperfecta, can be analysed. Also, healing of wounds, including chronic wounds, can be monitored. According to other examples, the method and apparatus according to the invention can be implemented in the study of the effect of cosmetic products, especially by observing the stimulation of collagen fibres after application of anti-aging products to the skin.

### BRIEF DESCRIPTION OF THE DRAWINGS

The invention will be better understood upon reading the following description, which is given solely by way of non-limiting example and is made with reference to the drawings in which:

FIG. 1 is a schematic illustration of the architecture of an apparatus for measuring a physical quantity representative of the elasticity of a material;

FIG. 2 is a schematic illustration of a stimulator and a measurement device of the apparatus of FIG. 1;

FIG. 3 is a flow chart of a method for measuring a physical quantity representative of the elasticity of a material using the apparatus of FIG. 1;

FIG. 4 is a three-dimensional graph illustrating displacement measurements acquired by the apparatus of FIG. 2:

FIG. 5 is a graph illustrating the calculation of the displacement velocity of a shear wave;

FIG. 6 is a three-dimensional graph illustrating, for a frequency component, displacement measurements acquired by the apparatus of FIG. 2;

FIG. 7 is a graph illustrating determination of a phase velocity of the frequency component of FIG. 6;

FIG. 8 is a graph illustrating damping of the frequency component of FIG. 6;

FIG. 9 is a graph representing a dispersion curve determined using the apparatus of FIG. 1;

FIG. 10 is a graph showing a profile of phase velocities determined using the apparatus of FIG. 1;

FIG. 11 is a graph illustrating tomography constructed by the apparatus of FIG. 2;

FIG. 12 is a graph showing damping of the frequency component of FIG. 6 as a function of depth;

FIG. 13 is a graph representing, on a same graphical representation, the phase velocities measured using the method of FIG. 3 and those determined using another known method for determining phase velocities.

In these figures, the same references are used to refer to the same elements. In the remainder of this description, characteristics and functions well known to the person skilled in the art are not described in detail.

### DETAILED DESCRIPTION

In this description, a detailed example of an embodiment is first described in a chapter I with reference to the figures. Then, in a chapter II, alternatives to this embodiment are set forth. Finally, the advantages of the different embodiments are set forth in a chapter III.

### Chapter I: Exemplary Embodiment

In the exemplary embodiment described hereinafter, the material measured is human skin. Of course, the apparatus and method as described can be implemented to measure other materials.

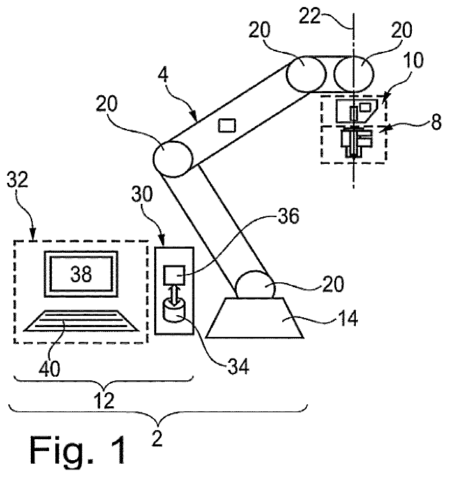

FIG. 1 represents a non-invasive apparatus 2 for measuring a physical quantity representative of elasticity of the skin. The apparatus 2 comprises:
a hinged arm 4,
a stimulator 8 and a measurement device 10, attached to a distal end of the arm 4, and
a computing processing unit 12 connected to the stimulator 8 and the measurement device 10.

A proximal end of the arm 4 is attached without any degree of freedom to a fixed support 14. The arm 4 comprises several hinges 20 which allow both the stimulator 8 and the measurement device 10 to be rotatably moved simultaneously about a projection axis 22. In FIG. 1, the axis 22 is vertical. Here, the hinges 20 allow the stimulator 8 and the measurement device 10 to be moved relative to the support 14 with six degrees of freedom.

Once the arm 4 has been deformed to place the stimulator 8 and measurement device 10 into the desired position, the arm 4 holds the stimulator 8 and measurement device 10 stationary in that position. The hinges 20 are, for example, manually actuated by a user or actuated by electric motors. The arm 4 also serves as a support for electrical conductors that connect the stimulator 8 and the measurement device 10 to the processing unit 12.

The stimulator 8 deforms the skin of a human being at an impact point when actuated. The impact point is located at the intersection of the projection axis 22 and the skin surface. The deformation of the skin produced by the stimulator 8 is such that it generates a shear wave which then propagates along the skin surface. This shear wave comprises frequency components at several different frequencies. Typically, in the case of human skin, the frequencies of these components are between 1 Hz and 3,000 Hz and, typically, between 1 Hz and 1,000 Hz.

The device 10 measures deformation of the skin surface, caused by the shear wave, at several measurement points aligned one behind the other along a measurement axis. This measurement axis extends parallel to a direction called the “measurement direction” in this text. Typically, the device 10 comprises more than three, ten or one hundred measurement points. Here, the device 10 comprises 400 measurement points. Hereinafter, the position of a measurement point along the measurement axis is marked by its abscissa x, for example expressed in mm or μm, measured from an origin O. Here, the measurement axis and the projection axis 22 intersect substantially at right angles. The origin O is taken to be the intersection point of these two axes.

The distance between the measurement point closest to the impact point and the measurement point furthest from the impact point is denoted as Lₘₐₓ. Here, by way of illustration, the distance Lₘₐₓ is equal to 7 mm. In this embodiment, the measurement points are evenly distributed along the measurement axis. The distance between two consecutive measurement points is therefore 17.5 μm.

The processing unit 12 is connected to the device 10 to acquire measurements of this device. More precisely, the unit 12 acquires over time, with a sampling frequency fₑ, the displacement measured at each of the measurement points. Hereinafter, the displacement measured at a measurement point of abscissa x at time instant is denoted as u(x, t). For example, here the frequency fₑ is equal to 8 kHz.

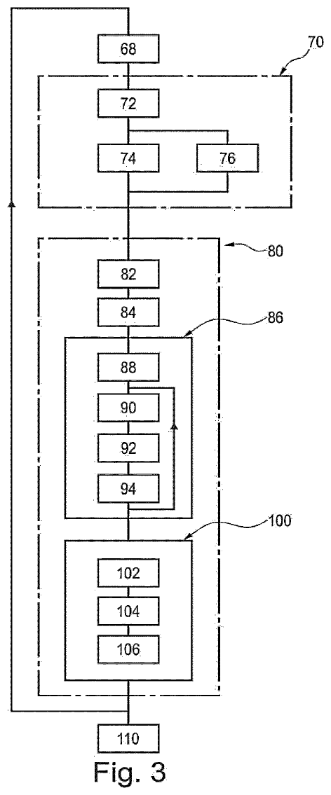

The unit 12 is able to process the signals u(x, t) thus acquired in order to extract therefrom a physical quantity representative of the elasticity of the skin at different depths. To this end, the unit 12 comprises a central processing unit 30 and a machine interface 32. The central processing unit 30 comprises:
a memory 34 comprising instructions for executing the method of FIG. 3, and
a programmable microprocessor 36 capable of executing the instructions stored in the memory 34.

The interface 32 allows the physical quantity measured representative of the elasticity of the skin to be displayed. Typically, to this end, the interface 32 comprises a screen 38. Here, the interface 32 also comprises a keyboard 40 for, for example, acquiring a control for triggering the execution of the measurement method of FIG. 3.

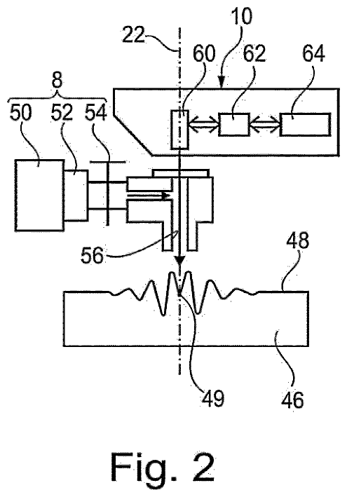

FIG. 2 represents the stimulator 8 and the measurement device 10 in more detail. In FIG. 2, the skin is schematically represented with the reference 46 and the surface of the skin 46 bears the reference 48. In this figure, the surface 48 is represented in a deformed form after being subjected to an impact applied by the stimulator 8. The impact point on the skin surface bears the reference 49.

To create the shear wave that propagates at the skin surface, the stimulator 8 uses, in this embodiment, a jet of air to impact the skin at the impact point 49. To this end, the stimulator 8 comprises:
a pressurised air tank 50,
a pressure reducing valve 52 fluidly connected to the tank 50,
a controllable solenoid valve 54, and
a nozzle 56 fluidly connected to an outlet of the solenoid valve 54.

For example, the air pressure contained in the tank 50 is greater than 0.6 or 0.8 MPa. Here, the pressure reducing valve 52 lowers the air pressure. For example, the air pressure at the outlet of the pressure reducing valve 52 is between 0.1 MPa and MPa or between 0.1 and 0.4 MPa.

The solenoid valve 54 can be moved from an open position to a closed position and vice versa under the control of the unit 12. In the closed position, the solenoid valve 54 prevents air from escaping from the tank 50. In the open position, on the contrary, the solenoid valve 54 allows air to escape from the tank 50. The air escaping from the tank 50 is then guided by the nozzle 56 to form a jet of air along the axis 22 which impacts the surface 48 of the skin 46 at the impact point 49.

The solenoid valve (54) allows the duration of the jet of air projected onto the surface 48 to be adjusted. Typically, the duration of the jet of air is less than 20 ms or 10 ms. Here, the duration of the jet of air is between 5 ms and 10 ms.

At least one part of the nozzle 56 extends along the axis 22 to direct the jet of air along the axis 22. The end of the nozzle 56, facing the surface 48, is mechanically separated from said surface, such that there is no direct mechanical contact between the apparatus 2 and the surface 48 in use thereof.

The measurement device 10 is an optical measurement device. In this embodiment, it comprises to this end:
an emitter 60 of a light beam that lights each measurement point,
a row 62 of optical sensors which sense the light reflected from each of the measurement points, and
a microprocessor 64 programmed to determine the displacement of the surface 48 at each of the measurement points from the reflected light sensed by each of the optical sensors.

Here, the device 10 is positioned relative to the stimulator 8 such that the measurement point closest to the impact point 49 is separated from that impact point by a distance greater than 0.5 mm or 0.8 mm and typically less than 5 mm. Preferably, the distance between the measurement point closest to the impact point and this impact point is between 0.7 mm and 1.3 mm or between 0.9 mm and 1.1 mm. Here, this distance is equal to 1 mm.

Such measurement devices are known and marketed. For example, in this embodiment, the device 10 is that marketed by the company KEYENCE® under the reference LJ-V 7020. In this case, the emitter 60 is a laser source which emits a monochromatic and collimated light beam. Thus, the device 10 is not described in more detail hereinafter.

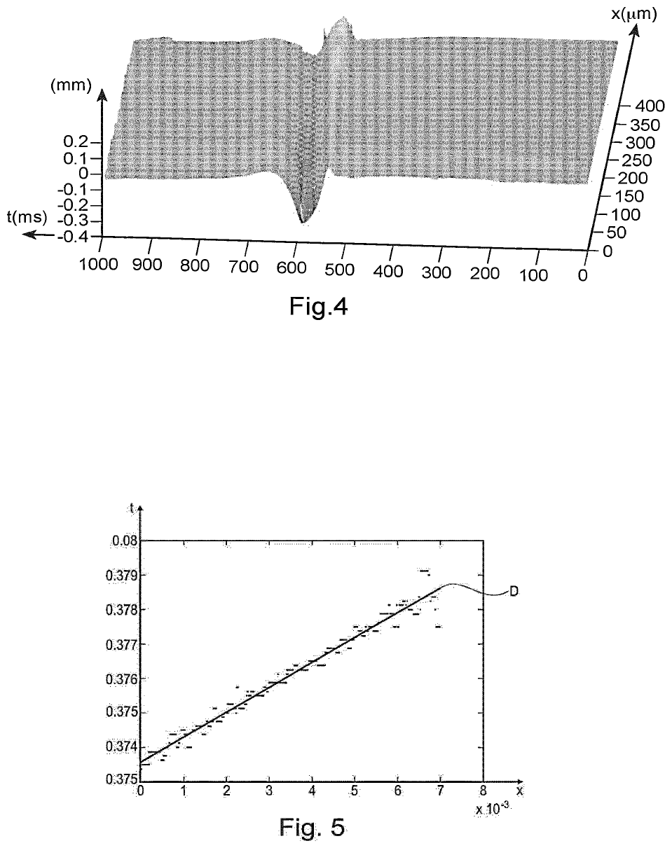

The operation of the apparatus 2 will now be described using FIG. 3 and with reference to the graphs of FIGS. 4 to 13.

In an initial step 68, the arm 4 is deformed to place the stimulator 8 and the measurement device 10 in proximity to a part of the human body covered with the skin to be studied. The apparatus 2 allows any part of the human body to be studied. For example, experimental results set forth in FIGS. 4 to 13 have been obtained on the forearm of a human being. Typically, the stimulator 8 and the device 10 are placed relative to the surface 48 of the skin such that the axis 22 of projection is at an angle of between 75° and 110°, and preferably between 80° and 100°, to the direction normal to the skin at the impact point 49.

The lower end of the nozzle 56, facing the surface 48, is separated from the impact point 49 by a distance greater than 1 mm or 2 mm or 5 mm, and generally less than 20 mm.

Once the apparatus 2 is correctly positioned with respect to the skin 46, a phase of acquiring the signals u(x, t) is carried out.

More precisely, once the stimulator 8 and the device 10 are correctly positioned relative to the surface 48, in a step 72, the unit 12 controls the stimulator 8 to cause the emission of a jet of air and deform the skin. Here, the unit 12 controls the solenoid valve 54 to generate this jet of air. This jet of air then impacts the skin at the impact point 49. This causes a brief deformation of the surface 48 at this impact point 49. This deformation of the surface 48 in turn generates a shear wave which propagates along the surface 48 in all directions, and thus especially along the measurement axis of the device 10.

In a step 74, the device 10 measures, at each of the measurement points, displacement of the surface 48 caused by the shear wave propagating at the surface of the skin.

In parallel, in a step 76, at each sampling time instant and for each measurement point, the unit 12 acquires the measurement from the device 10. Thus, the unit 12 acquires each of the signals u(x, t).

FIG. 4 represents, on a three-dimensional graph, an example of the signals u(x, t) acquired. In this graph:
the horizontal axis represents time in milliseconds,
the vertical axis represents amplitude, in millimetres, of the displacement of the surface 48, and
the depth axis represents, in μm, the position x of the measurement point along the measurement axis.

Once the various signals u(x, t) are acquired, the acquisition phase 70 ends and a phase 80 of processing the signals u(x, t) begins. Phase 80 is carried out by unit 12.

In a step 82, for each signal u(x, t), the unit 12 searches for and identifies the time instant tₘᵢₙ(x) at which the signal u(x, t) passes through its absolute minimum.

Then, in a step 84, the unit 12 determines the equation of the straight line D that best approximates the point cloud formed by the points of coordinates (x; tₘᵢₙ(x)). The equation of the straight line is as follows: t=μ₁x+μ₀. The coefficients μ₁ and μ₀ are those obtained by implementing the least squares method.

FIG. 5 represents the point cloud formed by the points of coordinates (x; tₘᵢₙ(x)) and the straight line D obtained by the least squares method.

The shear wave propagating in the skin 46 is a dispersive wave, that is, its phase velocity depends on the frequency. The phase velocity is therefore not the same for each frequency component of this shear wave. By phase velocity, it is meant here, unless otherwise stated, the phase velocity of the fundamental mode of the shear wave. The fundamental mode corresponds to the mode for which the amplitude of the displacement of the surface 48 is maximum. This phase velocity is denoted as Vᵢ for the frequency fᵢ, wherein the index i is an order number identifying the frequency fᵢ. Here, the frequencies are ranked from the lowest frequency, denoted as f₁, to the highest frequency denoted as f_{Pmax}.

In the case of the skin, it has been observed that the different frequency components that propagate in the skin 46 are typically of between 1 Hz and 3,000 Hz and, most often, between 1 Hz and 1,000 Hz or between 1 Hz and 500 Hz or between 1 Hz and 400 Hz. By way of illustration, here, the frequencies fᵢ are chosen in this interval ranging from 1 Hz to 1,000 Hz.

Of particular importance is the lowest frequency, denoted as fₘᵢₙ, for which there is a phase velocity. It has been observed that this smallest frequency fₘᵢₙ is generally between 1 Hz and 10 Hz. Thus, in this method, the sampling pitch of the frequencies fᵢ in the interval [1 Hz; 10 Hz] is chosen to be small, that is, here less than 2 Hz or 1 Hz. Conversely, in the interval [10 Hz, 1000 Hz], the sampling pitch of the frequencies fᵢ is chosen to be larger. For example, in this interval [10 Hz; 1,000 Hz], the sampling pitch is greater than 5 Hz or 10 Hz or 20 Hz. Thus, the frequencies fᵢ are separated from each other by a pitch of 1 Hz in the interval [1 Hz; 10 Hz] while they are separated from each other by a pitch greater than 5 Hz or 10 Hz in the interval [10 Hz; 1,000 Hz].

In a step 86, the unit 12 determines, for each selected frequency fᵢ, if any, the corresponding phase velocity Vᵢ.

For this, in an operation 88, the unit 12 filters each signal u(x, t) by means of a bandpass filter centred on the frequency fᵢ. The signal u(x, t) filtered at the frequency fᵢ is hereinafter denoted as uᵢ(x, t). The −3 dB bandwidth of this bandpass filter is between the frequencies fᵢ₋₁ and fᵢ₊₁. Typically, this bandwidth is less than 20 Hz or 10 Hz for the frequencies fᵢ in the interval [10 Hz; 1,000 Hz] and less than 2 Hz for the frequencies fᵢ in the interval [1 Hz; 10 Hz].

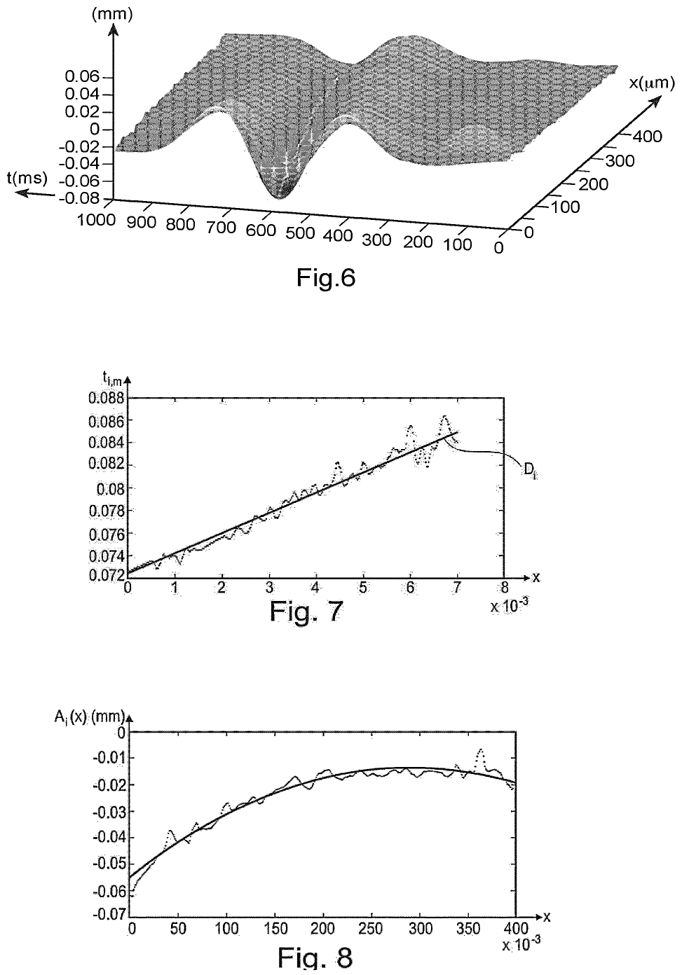

FIG. 6 represents different signals uᵢ(x, t) obtained by filtering, at a frequency of Hz, the signals u(x, t) of FIG. 4. This graph is identical to the graph of FIG. 4 except that it represents the signals uᵢ(x, t) and not the signals u(x, t).

In an operation 90, the unit 12 searches for and identifies, for each signal uᵢ(x, t), the time instant t_{i,m}(x) at which that signal passes through its absolute minimum. For this, if the time instant t_{i-1,m}(x) or t_{i+1,m}(x) has already been identified for the frequency fᵢ₋₁ or the frequency fᵢ₊₁, respectively, the unit 12 searches for the time instant t_{i,m}(x), as a priority, in a time interval centred on this time instant t_{i-1,m}(x) or t_{i+1,m}(x).

If no time instant t_{i-1,m}(x) or t_{i+1,m}(x) has previously been identified, then the time instant t_{i,m}(x) is searched for in a time interval centred on the time instant tₘᵢₙ(x) identified in step 82.

In an operation 92, the unit 12 calculates the phase velocity Vᵢ for the frequency fᵢ from the signals uᵢ(x, t). For this, the unit 12 determines the equation of the straight line Di that best approximates the point cloud formed by the points of coordinates (x; t_{i,m}(x)). The equation of the straight line Di is as follows: t_{i,e}(x)=μ_{i,1x}+μi,0. The coefficients μ_{i,1} and μ_{i,0} are obtained by implementing the least squares method as described for step 84.

FIG. 7 represents the point cloud of coordinates (x, t_{i,m}(x)) as well as the line Di.

Once the equation of the straight line Di is determined, the unit 12 also estimates the approximation error, that is, the deviation between points of coordinates (x; t_{i,m}(x)) and points of coordinates (x; t_{i,e}(x)) which have the same abscissa and lie on the straight line Di.

If this error exceeds a predetermined threshold errₘₐₓ, and then it is considered that there is no phase velocity for the frequency fᵢ because energy of the frequency component of frequency fᵢ of the shear wave is negligible. Here, for example, the approximation error is estimated using the following relation (1):

$${\sum }_{p = 1}^{{P}_{max}} {( {t}_{i , m} ( {x}_{p} ) - {μ}_{i , 1} {x}_{p} - {μ}_{i , 0} )}^{2} \leq {err}_{max} \qquad [ MATH 2 ]$$

where:
μ_{1,i} and μ_{i,0} are the coefficients of the straight line Di,
xₚ is equal to the position x of the p-th measurement point counted from the first measurement point closest to the impact point,
Pₘₐₓ is equal to the number of measurement points distributed along the measurement axis,
errₘₐₓ is a predetermined constant.

If this approximation error is less than or equal to the threshold errₘₐₓ, then the phase velocity Vᵢ is taken to be equal to 1/μ_{i,1}. The phase velocity Vᵢ thus obtained is the phase velocity of the fundamental mode at the frequency fᵢ. Indeed, it is only obtained from the minimums of the signals uᵢ(x, t), that is, from the points where the amplitude of the displacement is maximum.

In an operation 94, the unit 12 also determines the attenuation Aᵢ(x) of the frequency component at the frequency fᵢ. Here, the attenuation Aᵢ(x) is taken to be equal to the amplitude of the minimum of the signal uᵢ(x, t) identified in operation 90. Thus, in this embodiment, the attenuation Aᵢ(x) is equal to uᵢ(x; t_{i,m}(x)).

FIG. 8 represents the change in the attenuation Aᵢ(x) as a function of the position x for the signals uᵢ(x,t) of FIG. 4.

Steps 88 to 94 are reiterated for each of the frequencies fᵢ. Hereinafter, the smallest and largest of the frequencies fᵢ for which a phase velocity Vᵢ has been determined are denoted as fₘᵢₙ and fₘₐₓ, respectively. The curve formed by the set of points of coordinates (Vᵢ; fᵢ) is called the dispersion curve. FIG. 9 represents the dispersion curve obtained from the u(x,t) signals of FIG. 4.

The longer the wavelength λᵢ of a frequency component of the shear wave, the deeper said frequency component propagates under the surface 48 of the skin 46. Thus, the velocity Vᵢ of a frequency component of wavelength λᵢ represents the mechanical properties of the skin at a depth pᵢ. At this point, it is reminded that the phase velocity Vᵢ is related to the mechanical properties of the skin 46 by the following relationship: Vᵢ=(Eᵢ/(2ρᵢ(1+vᵢ))^{0.5}, where:
Eᵢ is the Young's modulus of the skin at depth pᵢ,
ρᵢ is the density of the skin at depth pᵢ, and
vᵢ is the Poisson coefficient of the skin at depth pᵢ.

Thus, considering that the density ρi and the coefficient vᵢ are known constants, the velocity Vᵢ is directly representative of the Young's modulus Eᵢ at depth pᵢ. Thus, the velocity Vᵢ is representative of the elasticity of the skin at depth pᵢ.

Therefore, the profile of velocities Vᵢ as a function of depth pᵢ corresponds to a cross-sectional view, along the measurement axis, of the mechanical properties of the skin.

In a step 100, the unit 12 transforms the dispersion curve into a profile of velocities as a function of depth pᵢ.

For this, in an operation 102, the unit 12 converts each frequency fᵢ into a corresponding wavelength λᵢ. Indeed, the unit 12 uses the following relationship (2): λᵢ=Vᵢ/fᵢ.

Then, in an operation 104, the unit 12 calculates the value of a coefficient α that converts each wavelength λᵢ into a corresponding depth pᵢ. Here, the depth pᵢ is the distance separating a point, buried under the skin, and the surface 48 of the skin. This coefficient α relates each wavelength λᵢ to the corresponding depth pᵢ according to the following relationship (3): pᵢ=αλᵢ.

This coefficient α is a constant for a given measurement axis. By contrast, in the case of anisotropic viscoelastic materials such as the skin, the coefficient α varies as a function of the measurement direction. In other words, the coefficient α depends on the direction in which the measurements are made.

Here, the coefficient α is calculated by the unit 12 using the following relationship (4): α=Zₘₐₓ/λₘₐₓ, where:
Zₘₐₓ is equal to half the distance Lₘₐₓ, and
λₘₐₓ is the largest of the wavelengths λᵢ obtained after the operation 102.

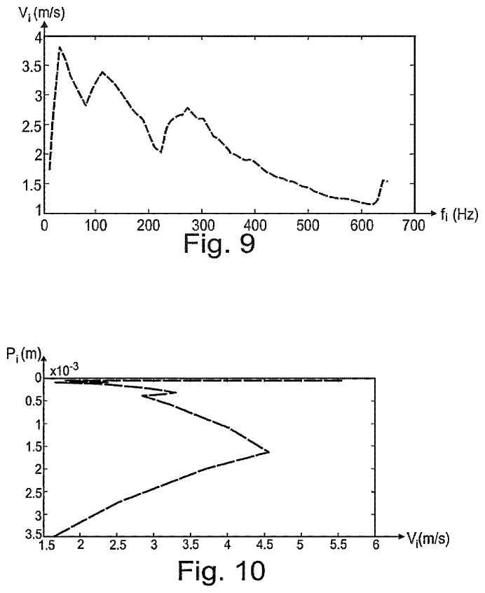

Once the value of the coefficient α is calculated, in an operation 106, the unit 12 converts each wavelength λᵢ into a depth pᵢ using the relationship (3) above. Thus, a frequency fᵢ corresponds to a wavelength λᵢ which in turn corresponds to a depth pᵢ. By replacing in each coordinate point (Vᵢ; fᵢ), the frequency fᵢ by the corresponding depth pᵢ, the profile of velocities measured by the apparatus 2 is obtained. An example of such a profile of velocities is represented in FIG. 10.

Here, step 68 and phases 70 and 80 are reiterated several times, each time rotating the measurement axis by a predetermined angle about the axis 22. For this, during each new iteration of step 68, the arm 4 is deformed so as to rotate the stimulator 8 and the measurement device 10 on themselves. This rotation does not modify the position of the projection axis 22, and thus the position of the impact point 49. For example, during each new iteration of step 68, the measurement axis is angularly offset from its previous position by at least 1° or 5°, and for example by 10° or 20°.

During each new iteration of the phase 80 of processing the data acquired, step 100 is again executed. Indeed, as previously explained, the value of the coefficient α highly depends on the direction of the measurement axis in the case of human skin.

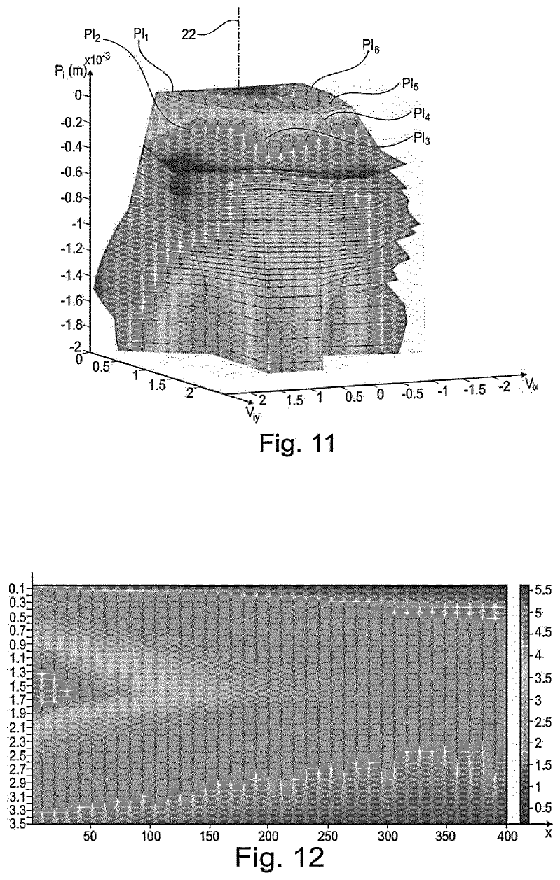

Finally, in a step 110, the mechanical properties of the skin as a function of the depth pᵢ are displayed on the screen 38. Different graphical representations are possible. For example, the profile of velocities, such as that represented in FIG. 10, is displayed on the screen 38. However, preferably, the different profiles of velocities obtained for different measurement directions are simultaneously displayed on a same graph to form a tomography of the skin 46 at the impact point 49. Such a tomography is represented in FIG. 11. In this tomography, the axis of ordinates represents the depth pᵢ. The axis 22 corresponds to the projection axis of the apparatus 2. Different measurement planes Pl₁ to Pl₆ are represented. These planes Pl₁ to Pl₆ are angularly offset from each other. Here, each of these measurement planes contains the axis 22 and extends parallel to a respective measurement direction. Each measurement plane contains the profile of velocities measured along the measurement axis parallel to that respective measurement direction. The axes of abscissas thus represent the coordinates Vᵢₓ and V_{iy} of the velocity Vᵢ measured.

FIG. 12 represents the attenuation Aᵢ(x) as a function of depth. In addition, the axis of abscissas represents the position x of the measurement point. The axis of ordinates represents the depth pᵢ in millimetres. The colour of each point of coordinates (x; p) codes attenuation of the velocity Vᵢ for that depth p and that abscissa x. For this, the depth is transformed into a corresponding wavelength λᵢ using the relationship (3), and then the wavelength λᵢ thus obtained is transformed into a corresponding frequency fᵢ using the relationship (2). In step 94, the attenuation Aᵢ(x) as a function of the position x has been plotted for all frequencies fᵢ, and thus for the particular frequency fᵢ corresponding to that depth p. This has been illustrated in the graph in FIG. 8. In the graph in FIG. 8, the attenuation Aᵢ(x) corresponding to the abscissa is plotted. It is the value of this measured attenuation Aᵢ(x) that is coded with a particular colour at the point of coordinates (x; p) in the graph in FIG. 12.

The different phase velocities Vᵢ can be determined from the signals u(x,t) by other methods, such as for example the method known as MASW (Multichannel Analysis of Surface Waves). The MASW method makes it possible to determine, for each frequency fᵢ, the velocity Vᵢ of the fundamental mode as well as the phase velocities of the modes of higher order than the order of the fundamental mode.

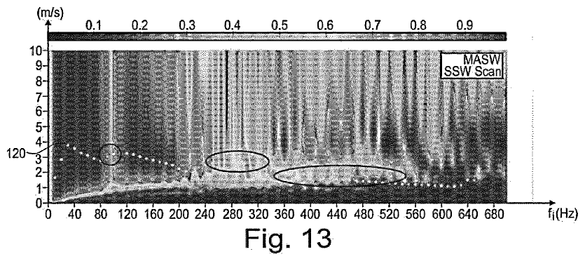

FIG. 13 represents on a same graph the phase velocity Vᵢ determined by the method of FIG. 3 and the different phase velocities determined by the MASW method. The axis of abscissas represents the frequency fᵢ, the axis of ordinates represents the amplitude of the determined phase velocity. The velocities Vᵢ determined by the method in FIG. 3 are represented by a dotted curve 120.

The phase velocities for a given frequency fᵢ as determined by the MASW method are coded with a colour which is darker the greater the amplitude of this phase velocity. For a same frequency fᵢ, the MASW method determines several phase velocities corresponding, respectively, to the fundamental mode and to the other higher order modes. From these determined phase velocities, that with the largest amplitude corresponds to the phase velocity of the fundamental mode. As shown in this graph, in most cases the velocity Vᵢ determined by the method in FIG. 3 passes through the darkest location in the graph in FIG. 13. This indicates that the phase velocities of the fundamental mode determined by the two different methods coincide.

By contrast, there are zones, surrounded by ellipses on the graph in FIG. 13, where this is not the case. This is particularly true for high frequencies, that is, frequencies above 240 Hz, but also for some lower frequencies. In these zones, the phase velocity of the fundamental mode, as determined by the MASW method, sharply drops and then rises again just as sharply. Hereinafter, these sudden drops in the phase velocity of the fundamental mode are called “phase jumps”. Such a phase jump is circled near 90 Hz in FIG. 13. Conversely, the method in FIG. 13 does not produce such phase jumps. In addition, the method in FIG. 13 allows the phase velocity of the fundamental mode to be determined at much higher frequencies than is possible by implementing the MASW method. Because of this, the method in FIG. 13 is considered to be more accurate than known methods.

### Chapter II: Alternatives

### Alternatives to the Measurement Device

In one particular alternative, the device is capable of measuring, simultaneously and along several measurement axes angularly offset from each other, the displacement of the surface of the skin or other material. Thus, with such a measurement device, it is not necessary to rotate it about the projection axis 22 or the number of rotations to be performed is smaller. For example, such a measurement device comprises an array of optical sensors for each measurement axis.

In another embodiment, the measurement axis does not pass through the impact point 49 but next to that impact point.

The device 10 may also be implemented using a camera that acquires images of the surface 48 at a high frequency.

Other embodiments of the stimulator 8 are possible. For example, in one alternative, the jet of air is replaced by a jet of another gas, such as for example carbon dioxide, or by a jet of liquid such as water.

The stimulator does not necessarily emit a jet of fluid to generate the shear wave at the surface of the material, or substrate, to be measured. Such a shear wave may also be generated by a stimulator directly contacting the material using utensil. For example, the stimulator may be a hammer which strikes the material at the impact point 49. It may also be a projectile such as a rubber ball or the like that is projected along the axis 22 to impact the material at the impact point 49.

The measurement device is not necessarily an optical measurement device. This is particularly true when the apparatus 2 is applied to a large surface area substrate where overall size restrictions are relaxed. For example, the signals u(x, t) can also be measured by arranging a displacement sensor directly on each of the measurement points. There are a large number of known displacement sensors likely to be suitable for such an application. For example, the displacement sensor may be an accelerometer.

In another embodiment, an element radiating electromagnetic waves or reflecting electromagnetic waves at a particular wavelength is arranged at each of the measurement points. The sensors of the measurement device measure the displacement of the surface at each of these measurement points from the electromagnetic radiation emitted or reflected at the measurement point.

### Alternatives to the Method

Other methods are possible for constructing the phase velocity of the fundamental mode as a function of depth. For example, as shown with FIG. 13, the method known by the acronym MASW can be applied although this is now considered to be less accurate. In another embodiment, one alternative to the MASW method is applied, this alternative having been modified to reduce the problem of phase jumps observed in FIG. 13. Finally, other methods than the MASW method have been developed in other technical fields such as, for example, geophysics and can be transposed here as long as these methods determine the phase velocity of the fundamental mode.

Alternatively, the sampling pitch of the frequencies fᵢ is the same over the whole analysis interval. For example, in the previous exemplary embodiment, the sampling pitch is the same over the entire interval ranging from 1 Hz to 1000 Hz.

Other methods for searching and identifying the time instant t_{i,m}(x) are possible. For example, the time instants t_{i,m}(x) are searched for without limiting the search to a predetermined time interval. In this case, the search is performed without taking account of the time instants t_{i-1,m}(x) or t_{i+1,m}(x) or tₘᵢₙ(x). Thus, steps 82 and 84 can be omitted.

The approximation error can be estimated differently. In particular, many other relationships are possible to calculate this approximation error. For example, the following relationship (5) can be used instead of relationship (1):

$${\sum }_{p = 1}^{{P}_{max}} {( {t}_{i , m} ( {x}_{p} ) - {μ}_{i , 1} {x}_{p} - {μ}_{i , 0} )}^{2} \leq {err}_{max} \qquad [ MATH 3 ]$$

In another embodiment, the coefficient α is not determined as a function of the signals u(x, t) measured by the device 10. For example, in a simplified embodiment, the coefficient α, used for a given measurement direction, is provided by the user of the device 12.

Instead of rotating the stimulator 8 on itself to make measurements in different measurement directions, it is also possible to keep the measurement direction constant and move the impact point along a straight line to progressively scan a portion of the material or substrate.

Measurements along a same measurement axis may be repeated at different time instants to see the change over time in these measurements. For example, this may be applied to measure the change over time in the mechanical properties of a material or substrate, for example the change over time in the mechanical properties of skin following the application of a moisturising product.

### Other Alternatives

Of course, and as previously mentioned, the apparatus 2 described herein may be applied to other viscoelastic and anisotropic materials than skin. For example, it can be applied to any similar viscoelastic material such as artificial skin. It can also be applied to animal skin, including marine animal skin such as fish skin.

The apparatus 2 can also be applied to other viscoelastic materials such as for example vegetable or fruit skin.

When the apparatus 2 is applied to other viscoelastic materials than skin, the interval of frequencies fᵢ for which the unit 12 calculates the velocity Vᵢ may be different from the interval [1 Hz; 1,000 Hz]. Similarly, if the viscoelastic material to which the apparatus 2 is applied is not anisotropic, the coefficient α can be calculated for a first measurement direction and the same value of this coefficient α is then used for other measurement directions angularly offset from the first measurement direction. In this case, for these other measurement directions, the operation 104 is not reiterated.

Other mechanical properties than the Young's modulus of the material or substrate at different depths can be deduced from the phase velocity Vᵢ. For example, the viscosity can also be estimated from the velocity Vᵢ.

The method in FIG. 3 for constructing the dispersion curve can be implemented in any non-invasive application for measuring a physical quantity representative of mechanical properties of a substrate. Indeed, as previously explained, this method allows a more accurate phase velocity of the fundamental mode to be obtained. For example, the method in FIG. 3 can also be implemented in a non-invasive apparatus for measuring mechanical properties in depth of a substrate such as a road surface or any multilayer structure. In the case of such substrates, it is not necessary to measure the profile of velocities in several different directions angularly offset from each other. Nor is it necessary to determine the value of the coefficient α for each of the measurement directions. Finally, in the case of substrates other than the skin, the stimulator 8 and the measurement device 10 are adapted to that substrate. For example, in the case of a road surface, the stimulator 8 is formed by a mass which impacts the road surface. In the case of a road surface, the device measures the shear wave over a distance typically greater than 7 mm.

Determining the value of the coefficient α as a function of the measurements of the measurement device can also be implemented in any other apparatus for measuring the mechanical properties of a material or substrate from a profile of phase velocities of the fundamental mode. Indeed, calculating the value of the coefficient α as previously described increases accuracy of the conversion of the dispersion curve into a profile of velocities.

### Chapter III: Advantages of the Embodiments Described

As the embodiments of the measurement apparatus and method have been described for measurements on human skin, technical advantages and effects listed below apply equivalently to the apparatus and method according to the invention when implemented with other viscoelastic and/or deformable materials or substrates.

The measurement method described herein allows the phase velocity of the fundamental mode of the shear wave to be measured at different depths and not just at the surface. It therefore allows mechanical properties of a material or substrate to be revealed at different depths under the surface of the material or substrate and not just at the surface. In addition, the apparatus 2 allows mechanical properties to be revealed at different depths while remaining non-invasive, that is, without the need for incision of the material or substrate.

For measurements on human skin, calculating the value of the coefficient α from the measured signals u(x, t) and more precisely from the dispersion curve, allows the value of the coefficient α to be automatically adapted to the skin on which the measurement is made and to the measurement direction chosen. Indeed, in contrast to other substrates, it has been observed that the value of the coefficient α significantly varies from one human being to another and also significantly varies depending on the measurement direction chosen. Thus, the automatic calculation of the value of the coefficient α improves the accuracy of the profile of phase velocities. Thus, a better accuracy on the depth of observation is obtained.

Constructing a profile of phase velocities for several directions angularly offset from each other makes it possible to generate a tomography of a mechanical property of the measured skin or material. Such a tomography especially allows anisotropy of this mechanical property to be observed as a function of the measurement direction. It is thus possible to quantify tensile forces in all directions measured in depth, and this tension can be represented in three dimensions. For example, healing of wounds can be analysed and monitored by measuring tensile forces, which indicate cell activity.

Calculating the phase velocity of the fundamental mode from the time instants t_{i,m}(x) and the positions x where the minimum of the signal uᵢ(x, t) occurs, makes it possible to accurately determine phase velocity of the fundamental mode at the frequency fᵢ. This therefore increases accuracy of the constructed profile of phase velocities, and hence accuracy of the measurement of the mechanical properties of the material or substrate.

Automatically identifying the frequency fₘᵢₙ makes it possible to increase reproducibility of the measurement method as this minimum frequency is not manually determined by the user. Additionally, when the condition (1) is used, this allows the wavelength λₘₐₓ to be determined more accurately, which also improves the accuracy of the determination of the value of the coefficient α, and thus ultimately the accuracy of the profile of phase velocities.

Automatically identifying the frequency fₘₐₓ avoids determining phase velocities for frequency components of the shear wave that do not exist or are negligible. This therefore improves the accuracy of the profile of phase velocities constructed by the apparatus 2.

1-12. (canceled)

13. A non-invasive method for measuring a physical quantity representative of the elasticity of a material, the method comprising the following steps of:
a) deforming the material at an impact point using a stimulator to generate a shear wave comprising components at different frequencies, these various components propagating at the surface of the material and causing a displacement of the surface of the material,
b) measuring, using a measurement device, the displacement of the surface of the material over time at at least three measurement points aligned one behind the other along a measurement axis,
wherein the method also comprises the following steps of:
c) determining the phase velocities of the fundamental mode of the various components of the shear wave generated, along the measurement axis, from the measurements of the measurement device, the set of pairs, each formed by a frequency fᵢ and the phase velocity Vᵢ of the fundamental mode determined for said frequency fᵢ, forming a dispersion curve of the fundamental mode in a measurement direction parallel to the measurement axis, wherein the index i is an order number of the frequency fᵢ and of the phase velocity Vᵢ, and
d) transforming said dispersion curve into a profile of phase velocities as a function of depth, the phase velocity at a given depth being a physical quantity representative of the elasticity of the material at said depth.

14. The method according to claim 13, wherein transforming the dispersion curve into a profile of phase velocities in the measurement direction comprises the following operations of:
1) converting each frequency fᵢ of the dispersion curve, into a corresponding wavelength λᵢ using the following relationship: λᵢ=Vᵢ/fᵢ, and then
2) calculating the value of a coefficient α for said measurement direction using the following relationship: α=Zₘₐₓ/λₘₐₓ, where:
Zₘₐₓ is equal to half the distance separating the two furthest measurement points along the measurement axis, and
λₘₐₓ is the largest of the wavelengths λᵢ obtained after executing the operation 1) for said measurement direction,
3) converting each wavelength λᵢ obtained after executing operation 1) into a corresponding depth pᵢ using the following relationship: pᵢ=αλᵢ, where α is the coefficient calculated in the operation 2).

15. The method according to claim 14, wherein the method comprises performing steps a), b), c) and d) for at least a first and a second measurement axes angularly offset from each other, said first and second measurement axes passing through the same impact point.

16. The method according to claim 13, wherein step c) of determining the phase velocities of the fundamental mode comprises reiterating the following operations for several different frequencies fᵢ:
for each measurement point:
filtering, using a bandpass filter centred on the frequency fᵢ and whose −3 dB bandwidth is between the frequencies fᵢ₋₁ and f₊₁, the signal u(x,t), measured at the measurement point of coordinate x along the measurement axis by the measurement device, to obtain a filtered signal uᵢ(x,t),
identifying a time instant t_{i,m}(x) at which the filtered signal uᵢ(x,t) passes through its absolute minimum, and then
calculating the velocity Vᵢ of propagation of this minimum along the measurement axis from the time instants t_{i,m}(x) and the positions x at which this minimum occurs, the velocity Vᵢ thus calculated being the phase velocity of the fundamental mode at said frequency fᵢ.

17. The method according to claim 16, wherein step c) comprises:
an operation of automatically identifying, among the set of frequencies fᵢ, a minimum frequency fₘᵢₙ below which the frequencies fᵢ no longer verify the following condition (1):
$${\sum }_{p = 1}^{{P}_{max}} {( {t}_{i , m} ( {x}_{p} ) - {μ}_{i , 1} {x}_{p} - {μ}_{i , 0} )}^{2} \leq {err}_{max}$$
where:
μ_{i,1} and μ_{i,0} are the coefficients of the straight line, determined by the least squares method, best approximating the points of coordinates (x; t_{i,m}(x)),
xₚ is equal to the position x of the p-th measurement point counted from the first measurement point closest to the impact point,
Pₘₐₓ is equal to the number of measurement points distributed along the measurement axis,
errₘₐₓ is a predetermined constant,
and then only the frequencies fᵢ greater than or equal to said frequency fₘᵢₙ are retained to form the dispersion curve.

18. The method according to claim 17, wherein step c) comprises:
an operation of automatically identifying, from the set of frequencies fᵢ, a maximum frequency fₘₐₓ above which the frequencies fᵢ no longer verify the condition (1), this operation of automatically identifying the frequency fₘₐₓ being carried out by testing this condition (1) for several frequencies fᵢ, and then only the frequencies fᵢ lower than or equal to said frequency fₘₐₓ are retained to form the dispersion curve.

19. The method according to claim 13, wherein all frequencies fᵢ are between 1 Hz and 3,000 Hz.

20. A non-invasive apparatus for measuring a physical quantity representative of the elasticity of a material, for implementing a method in accordance with claim 13, the apparatus comprising:
a stimulator capable of deforming the material at an impact point to generate a shear wave comprising components at different frequencies, these various components propagating at the surface of the material and causing a displacement of the surface of the material,
a measurement device capable of measuring the displacement of the surface of the material over time at at least three measurement points aligned one behind the other along a measurement axis,
wherein the apparatus comprises a processing unit capable of:
determining the phase velocities of the fundamental mode of the various components of the shear wave generated along the measurement axis from the measurements of the measurement device, the set of pairs, each formed by a frequency fᵢ and the phase velocity Vᵢ of the fundamental mode determined for said frequency fᵢ, forming a dispersion curve of the fundamental mode in a measurement direction parallel to the measurement axis, wherein the index i is an order number of the frequency fᵢ and the phase velocity Vᵢ, and
transforming said dispersion curve into a profile of phase velocities as a function of depth, the phase velocity at a given depth being a physical quantity representative of the elasticity of the material at said depth.

21. The apparatus according to claim 20, wherein the processing unit is configured to execute the following operations to transform the dispersion curve into a profile of phase velocities in the measurement direction:
1) converting each frequency fᵢ of the dispersion curve, into a corresponding wavelength λᵢ using the following relationship: λᵢ=Vᵢ/fᵢ, and then
2) calculating the value of a coefficient α for said measurement direction using the following relationship: α=Zₘₐₓ/λₘₐₓ, where:
Zₘₐₓ is equal to half the distance separating the two furthest measurement points along the parallel measurement axis, and
λₘₐₓ is the largest of the wavelengths λᵢ obtained after executing the operation 1) for said measurement direction,
3) converting each wavelength λᵢ obtained after the operation 1) into a corresponding depth pᵢ using the following relationship: pᵢ=α*λᵢ, where α is the coefficient calculated in the operation 2).

22. The apparatus according to claim 20, wherein:
the measurement device comprises an array of sensors each capable of measuring the amplitude of the deformation of the surface of the material at a respective measurement point, this array comprising at least three sensors each of which measures displacement of the surface of the material at three respective measurement points aligned one behind the other along a measurement axis, and
the apparatus comprises a hinged arm to which the sensor array is mounted, said hinged arm being capable of rotating the sensor array through a predetermined angle about an axis of rotation, to align the measurement axis of the sensor array with a first measurement axis and, alternately, with a second measurement axis angularly offset from the first measurement axis.

23. The apparatus according to claim 22, wherein:
the sensor array comprises:
a row of optical sensors that sense light reflected from each measurement point, and
a microprocessor configured to determine displacement of the surface of the material at each measurement point lit from the reflected light sensed by the row of optical sensors,
the measurement device comprises an emitter of a light beam which lights each measurement point aligned along the measurement axis.

24. The apparatus according to claim 20, wherein the stimulator is capable of projecting a jet of fluid onto the surface of the material which causes the material to deform at the impact point.
